(19) United States
(12) Patent Application Publication
(10) Pub. No.: US 2008/0183249 A1
(43) Pub. Date: Jul. 31, 2008

(54) ORAL CARE DEVICE

(76) Inventors: Tadanobu Kitagawa, Higashiazai-gun (JP); Yoshihiro KITAMURA, Hikone-shi (JP); Junichi HOSHINO, Omihachiman-shi (JP)

Correspondence Address: Cheng Law Group, PLLC, 1100 17th Street, N.W., Suite 503, Washington, DC, 20036, US

(21) Appl. No.: 12/014,487

(22) Filed: Jan. 15, 2008

(30) Foreign Application Priority Data
Jan. 31, 2007 (JP) 2007-021976

(51) Int. Cl. A61N 1/26; A46B 13/00

(52) U.S. Cl. 607/79; 15/22.1

(57) ABSTRACT

An oral care device has a handle with a touch electrode and a brush handle with a brush electrode for flowing a minute current into an oral cavity of a user by way of the user's body for removing plaque. The touch electrode is made of a non-metal material filled with carbon filaments, and is free from any metal elution or corrosion while it is in contact with the user's hand, which assures to keep the oral care device in good appearance and performance over a prolonged use.

### TECHNICAL FIELD

The present invention relates to an oral care device supplying an electric current to an oral cavity for removal of tooth plaque.

### BACKGROUND ART

Japanese patent publication No. 9-140453 discloses an electric toothbrush configured to flow an electric current to teeth for removal of tooth plaque. The toothbrush has a handle and a brush head extending from the handle. The handle is provided with a touch electrode for contact with a user's hand, while the brush head is provided with a conductor held in an electrical contact with a brush. A battery is included in the handle to give a potential difference between the touch electrode and the conductor so as to flow an electric current to the oral cavity through a user's body for removing the tooth plaque. The touch electrode is made of a metal and is exposed on the exterior of the handle for electrical contact with the user's hand. During a prolonged use, however, the metal-made touch electrode suffers from elution of metal ions or corrosion, which might be somewhat harmful or deteriorate the appearance of the toothbrush, or even fail to flow the current consistently.

### DISCLOSURE OF THE INVENTION

In view of the above problem, the present invention has been accomplished to provide an oral care device of which touch electrode is prevented from elution or corrosion for prolonged use without deterioration of the appearance and performance. The oral care device in accordance with the present invention includes a handle provided with a touch electrode for contact with a user's hand, a brush head extending from the handle and carrying a brush configured to be placed into an oral cavity of the user. The brush head is provided with a negative brush electrode disposed adjacent the brush and being configured to come into electrical contact with portions or substances in the oral cavity. A power source is accommodated within the handle to apply a voltage difference between the touch electrode and the brush electrode in order to flow a current within the oral cavity. The touch electrode is made of a non-metal material filled with carbon filaments. Accordingly, the touch electrode is free from any metal elution or corrosion while it is in contact with the user's hand to flow the current, which assure to keep the oral care device in good appearance and performance over a prolonged use.

Most preferably, the touch electrode is charged positive with respect to the brush electrode to charge the user's teeth positive, thereby attracting negative ions to the surface of the teeth for effectively decomposing and removing plaque therefrom. In this instance, the brush electrode is charged negative and is well prevented from elution in the oral cavity even when it is made of a metallic material.

Preferably, the handle is configured to have a water-tight interior space within which the power source is disposed together with terminals respectively for electrical connection with said brush electrode and said touch electrode. The touch electrode is configured to have a portion which extends continuously into the interior space for electrical contact with a corresponding one of the terminals. Thus, the touch electrode can be held in direct electrical contact with the corresponding terminal, while the electrical connection can be concealed within the handle to be water-proofed.

In this connection, the touch electrode is preferred to be molded as an integral part of the handle so that the handle can be easily shaped into a water-tight structure. Also with the integral molding, the handle can be free from any noticeable gap around the touch electrode, eliminating a possibility that any ionized material is trapped in the gap and therefore keeping the handle fee from contamination of the ionized material and associated lowering of the current flow from the touch electrode.

The touch electrode may be composed of a non-electrically conductive matrix within which said carbon filaments are dispersed. The touch electrode is electrically connected to the corresponding terminal by means of an electrically conductive adhesive. In this instance, the adhesive can permeate over the surface the conductive matrix for successful contact with the carbon fibers, thereby assuring a good electrical interconnection. Preferably, the adhesive is prepared in the form of a tap for easy connection of the touch electrode to the terminal. A screw may be used in combination with the tape to fasten the touch electrode to the terminal. In this case, the screw head is disposed in a recess formed in the outer surface of the touch electrode, and is concealed therein by a cap fitted in the recess.

Alternatively, the touch electrode may be electrically connected to the corresponding terminal by means of the screw without the use of the adhesive. In this instance, the tightening of the screw develops a force of squeezing the carbon fibers for electrical contact with the terminal, thereby assuring reliable electrical connection between the touch electrode and the terminal. Also in this case, the touch electrode may be formed with a recess for concealing therein the screw head.

These and still other advantageous features of the present invention will become apparent from the following detailed description of the embodiments when taken in conjunction with the attached drawings.

### BRIEF DESCRIPTION OF THE DRAWINGS

FIG. 1 is a vertical section of an oral care device in accordance with a first embodiment of the present invention;

FIG. 2 is a vertical section of an oral care device in accordance with a second embodiment of the present invention;

FIG. 3 is a vertical section of an oral care device in accordance with a third embodiment of the present invention;

FIG. 4 is a partial section of an oral care device in accordance with a fourth embodiment of the present invention;

FIGS. 5A and 5C are enlarged sectional views respectively illustrating aspects of the above embodiments;

FIG. 6 is a partial section of an oral care device in accordance with a fifth embodiment of the present invention;

FIG. 7 is an enlarged sectional view illustrating an electrical connection between a touch electrode and a corresponding terminal of the above embodiment;

FIG. 8 is partial section of an oral care device in accordance with a sixth embodiment of the present invention;

FIG. 9 is an enlarged sectional view illustrating an electrical connection between a touch electrode and a corresponding terminal of the above embodiment.

### BEST MODE FOR CARRYING OUT THE INVENTION

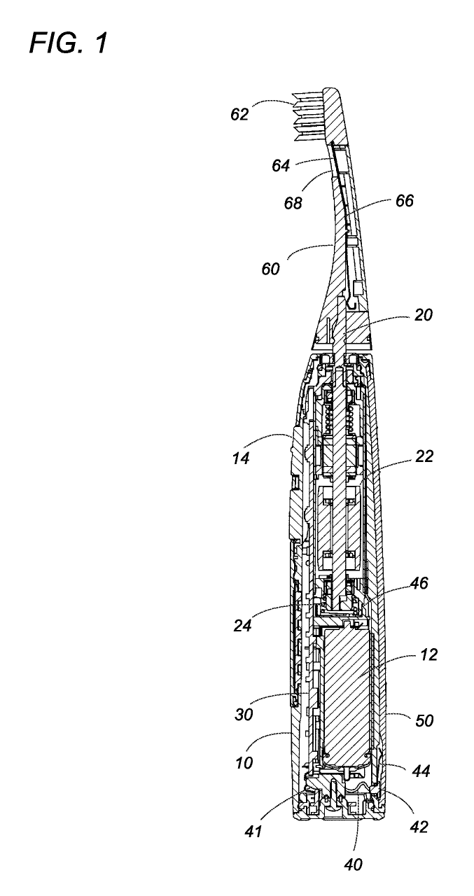

Referring now to FIG. 1, there is shown an oral care device in accordance with a first embodiment of the present invention. The oral care device is provided as an electric toothbrush having an oscillating brush and having a function of flowing minute current to an oral cavity of a user for removing plaque, although the present invention is not limited to this particular device.

The device includes a tubular handle 10 and a brush head 60 extending from the handle 10 and carrying the brush 62 at its front end. The handle 10 is provided with an oscillating shaft 20 which driven by an electromagnetic actuator 20 mounted in the handle 10 together with a battery 12. The actuator 22 is powered by the battery 12 and is configured to selectively oscillate the shaft 20 along its axis and about its axis at a rapid cycle of about 30,000 per minute. The selection is made by a selector 14 on the handle 10. The shaft 20 extends from the upper end of the handle 10 for detachable engagement into a slot 61 at the rear end of the brush head 60 so that the brush 60 is driven by the shaft 20 to oscillate for cleaning the teeth.

A circuit board 30 is mounted within the handle 10 to give a boosting circuit which boots a voltage of the battery for driving the actuator 20 as well as a current limiting circuit which generates a minute current of 30 μA to 100 μA from the battery 12.

The handle 10 is formed on its exterior surface with a touch electrode 50 for contact with a user's hand. The touch electrode 50 is made of a non-metal material filed with carbon fibers, and is cooperative with the handle 10 to define a water-tight interior space for accommodating therein the actuator 22, the battery 12, the circuit board 30, and associated members. The touch electrode 50 is electrically connected to the current limiting circuit so as to be positively charged with regard to a brush electrode 64 disposed in the brush head 60 adjacent to the brush 62. The brush electrode 64 is defined at a front end of a conductor 66 extending in the brush head 60, and is exposed in an opening 68. The conductor 66 has its rear end in electrical contact with the end of the shaft 20 and is connected to the current limiting circuit through the shaft 20 of electrically conductive material. Thus, the current limiting circuit flows the minute current through a path of the touch electrode 50, the user's hand, gums, teeth, sputum, toothpaste, and the brush electrode 64 for removing the plaque.

The electrical connection of the touch electrode 50 to the current limiting circuit is made through a terminal 40, a pin 42, and a contact member 44. The terminal 40 is in the form of a spring leaf supported to a block 41 fixed to the bottom of the handle 10 and is connected at its one end to the current limiting circuit. The other end of the terminal 40 is pressed against the pin 42 fitted in the wall of the handle 10. The contact member 44 extends from the pin for pressed contact with the back of the touch electrode 50 for establishing the electrical connection of the touch electrode to the current limiting circuit. The shaft 20 is electrically connected to the current limiting circuit by use of a coil spring 24 which is fixed at Its one end to the rear end of the shaft 20 and is connected at the other end to a terminal 46 leading to the current limiting circuit on the circuit board 30.

The touch electrode 50 includes the carbon fibers which are dispersed in a non-electrically conductive resin such as ABC resin to give electrical conductivity for electrical connection with the terminal 40 in the handle 10. Since the touch electrode 50 is devoid of any metal, it is free from elution of metallic ions while being kept in contact with the user's hand, and is therefore free from contamination of such metallic ions.

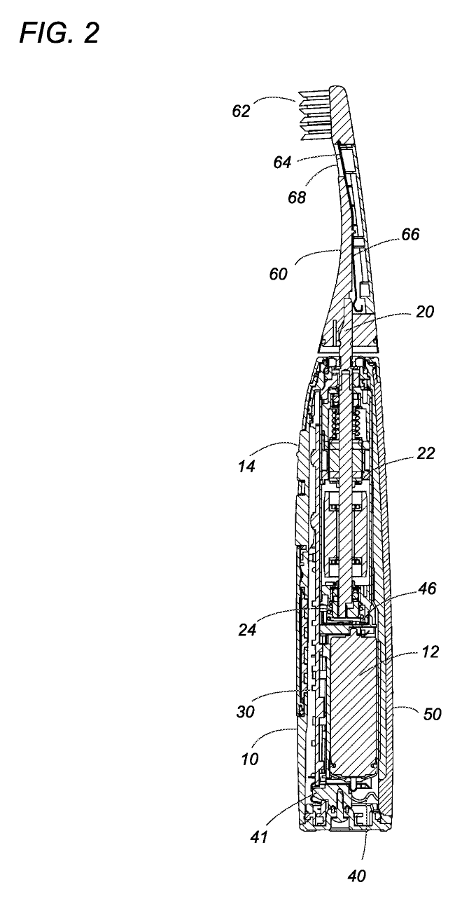

FIG. 2 shows an oral care device in accordance with a second embodiment of the present invention which is identical to the above embodiment except that the touch electrode 50 is molded together with the handle 10 to give a unitary molded structure. Like parts are designated by like reference numerals and duplicate explanation is deemed necessary. The touch electrode 50 is made of the non electrically conductive resin such as ABS resin dispersed with the carbon fibers, and is molded integrally with the handle 10 made of ABS resin such that a portion of the touch electrode 50 extends into the water-tight interior space for direct contact with the terminal 40 at the bottom of the handle 10. The touch electrode 50 extends in an overlapping relation with a rear wall of the handle 10 except at a bottom end of the handle 10 so to be exposed to the interior space thereat.

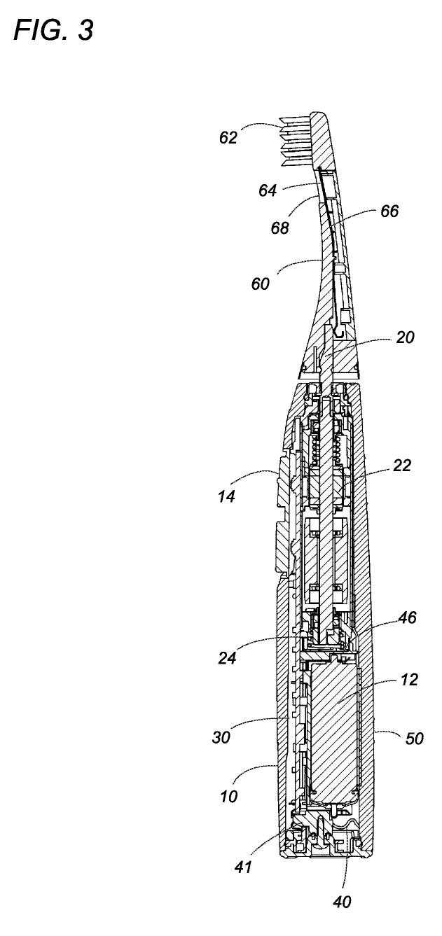

FIG. 3 shows a modification of the second embodiment of FIG. 2 in which the touch electrode 50 is molded to constitute the wall of the handle 10 to be entirely exposed to the interior space for contact with the terminal 40.

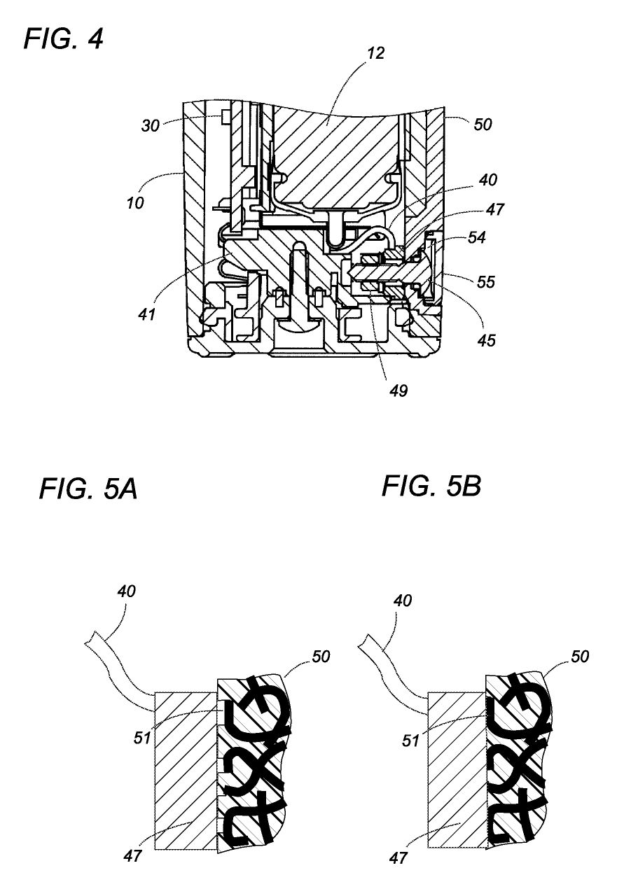

FIG. 4 shows another modification which is identical to the second embodiment of FIG. 2 except that the touch electrode 50 is fastened to the terminal 40 by use of a screw 45. The screw 45 extends through a bottom of a recess 54 formed in the outer surface of the touch electrode 50 into a nut 49 to hold a ring 47 between the nut and the touch electrode 50. The ring 47 is made of an electrical conductive material connected to the terminal 40 and is held in pressed contact with the touch electrode 50 to make the electrical connection thereat. Therefore, even if the carbon fibers 51 happen to be receded from the rear surface of the touch electrode 50, as shown in FIG. 5A, the tightening of the screw can squeeze the touch electrode 30, i.e., the non-electrical resin matrix to bring the carbon fibers 51 into intimate contact with the ring 47, as shown in FIG. 5B, to make a reliable electrical connection. The recess 54 is fitted with a cap 55 of a non-electrical material for concealing the screw head within the recess 54. The screw may be made of a non-electrical conductive material or of an electrically conductive material.

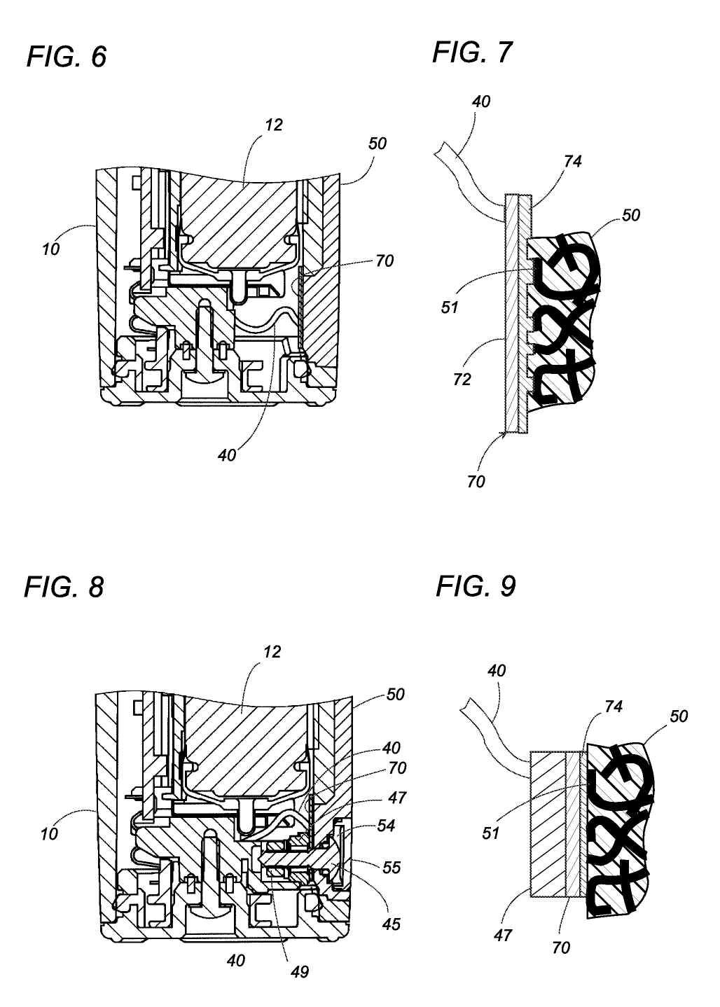

FIGS. 6 and 7 show a further modification which is identical to the second embodiment of FIG. 2 except for the use of an electrically conductive adhesive in the form of a tape 70 connected to the terminal 40. The tape 70 is composed of a film 72 of electrically conductive material leading from the terminal 40, and a viscous adhesive coating 74 also of an electrically conductive material. As shown in FIG. 5 the viscous adhesive coating can permeate into possible cavities of the touch electrode 50 for successful contact with the carbon fibers 51 even if the carbon fibers happen to be hidden in the cavities as illustrated.

FIGS. 8 and 9 show a still further modification which is similar to the above modification of FIG. 4 but additionally use the tape 70 as utilized in the above modification of FIG. 6. Like parts are designated by like reference numerals and duplicate explanations are omitted. The adhesive tape 70 is held between the ring 47 and the touch electrode 50. Thus, as shown in FIG. 9, the tightening of the screw 45 forces the viscous adhesive coating 74 to permeate into possible cavities of the touch electrode 50 for successful contact with the carbon fibers 51 even if the carbon fibers happen to be hidden in the cavities as illustrated.

Although the above embodiments and modifications are illustrated to have the positively charged touch electrode 50 with respect to the brush electrode 64, the present invention is not limited to this specific arrangement and may be designed to charge the touch electrode 50 negative with respect to the brush electrode, in consideration of that the touch electrode 50 is likely to be wetted during the use and would certainly suffer from elution if the touch electrode 50 is made of a metallic material.

1. An oral care device comprising:
a handle provided with a touch electrode for contact with a user's hand;
a brush head extending from said handle and carrying a brush configured to be placed into an oral cavity of a user, said brush head being provided with a brush electrode being disposed adjacent to said brush and configured to come into electrical contact with portions or substances in the oral cavity; and
a power source accommodated in said handle to apply a voltage difference between said touch electrode and said brush electrode;
characterized in that said touch electrode is made of non-metal material filled with carbon filaments.

2. An oral care device as set forth in claim 1, wherein said touch electrode is charged positive with respect to said brush electrode.

3. An oral care device as set forth in claim 1, wherein
said handle has a water-tight interior space within which said power source is disposed together with terminals respectively for electrical connection with said brush electrode and said touch electrode, and
said touch electrode has a portion which extends continuously into said interior space of said handle for electrical connection with the corresponding terminal.

4. An oral care device as set forth in claim 1, wherein
said touch electrode is molded as an integral part of said handle.

5. An oral care device as set forth in claim 4, wherein
said handle has a water-tight interior space within which said power source is disposed together with terminals respectively for electrical connection with said brush electrode and said touch electrode;
said touch electrode is composed of a non-electrically conductive matrix within which said carbon filaments are dispersed,
said touch electrode being electrically connected to a corresponding one of said terminals by means of an electrically conductive adhesive.

6. An oral care device as set forth in claim 5, wherein
said electrically conductive adhesive is configured into a tape,
said touch electrode is fastened to said terminal by means of a screw.

7. An oral care device as set forth in claim 6, wherein
said screw has a screw head disposed in a recess formed in a top surface of said touch electrode,
said recess is fitted with a cap for concealing the screw head.

8. An oral care device as set forth in claim 4, wherein
said handle has a water-tight interior space within which said power source is disposed together with terminals respectively for electrical connection with said brush electrode and said touch electrode;
said touch electrode is composed of a non-electrically conductive matrix within which said carbon filaments are dispersed,
said touch electrode being electrically connected to a corresponding one of said terminals by means of a screw extending from said touch electrode to said terminal.

9. An oral care device as set forth in claim 8, wherein
said screw has a screw head disposed in a recess formed in a top surface of said touch electrode, and
said recess is fitted with a cap for concealing the screw head.
